(19) United States
(12) Patent Application Publication
(10) Pub. No.: US 2024/0209429 A1
(43) Pub. Date: Jun. 27, 2024

(54) MONOCLONAL CLUSTERING USING DOUBLE STRANDED DNA SIZE EXCLUSION WITH PATTERNED SEEDING

(71) Applicant: Illumina, Inc., San Diego, CA (US)

(72) Inventors: Aathavan Karunakaran, Berkeley, CA (US); David Bracher, San Diego, CA (US)

(73) Assignee: Illumina, Inc., San Diego, CA (US)

(21) Appl. No.: 18/531,285

(22) Filed: Dec. 6, 2023

Related U.S. Application Data
(60) Provisional application No. 63/430,936, filed on Dec. 7, 2022.

(51) Int. Cl. C12Q 1/6855; B01L 3/00

(52) U.S. Cl. CPC C12Q 1/6855; B01L 3/502761; B01L 2200/0647; B01L 2200/16

(57) ABSTRACT

This application relates to methods of monoclonal clustering. In some examples, the monoclonal clustering utilizes double-stranded DNA.

### CROSS-REFERENCE TO RELATED APPLICATION

This application claims the benefit of U.S. Provisional Patent Application No. 63/430,936 filed Dec. 7, 2022, and entitled “Monoclonal Clustering using Double Stranded DNA Size Exclusion with Patterned Seeding,” the entire contents of which are incorporated by reference herein.

### FIELD

This application relates to methods of monoclonal clustering.

### BACKGROUND

Polyclonal clusters suffer from increased noise and lower signal due to the interference of the non-dominant template strands with signal from the primary template population of the cluster. The resultant reduction in signal and increase in noise ultimately leads to reduced basecalling performance, even after application of chastity filtering. The ability to create a higher fraction of monoclonal clusters is hindered by the Poissonian nature of the typical seeding process. In addition, the non-dominant strands are lost in the final readout, making the sequencing process inefficient at reading the available DNA on the flow cell. If seeding is done at low density to improve the percent of occupied clusters that are monoclonal, then the overall occupancy percentage is lowered, thereby reducing throughput.

### SUMMARY

Examples provided herein are related to methods and compositions for amplifying a target nucleic acid through seeding double-stranded DNA (dsDNA) onto a flow cell. In some examples, the flow cell includes a protein, and the dsDNA is connected to a ligand that interacts with the protein. In some examples, the flow cell includes a plurality of lawn primers immobilised on the flow cell.

Some examples herein provide a method of amplifying a target nucleic acid, the method includes seeding double-stranded DNA (dsDNA) onto a flow cell that includes a first portion that includes a protein, and a second portion that includes a plurality of lawn primers immobilised on the flow cell, wherein the dsDNA includes a target nucleic acid sequence and a complementary nucleic acid sequence, and wherein the dsDNA is connected to a ligand that causes an interaction between the ligand and the protein; after interaction between the ligand and the protein, denaturing the dsDNA to remove the complementary nucleic acid sequence; and amplifying the target nucleic acid sequence using the lawn primers.

In some examples, the plurality of lawn primers are capped on one end with positive charges. In some examples, the positive charges cause diffusion of the dsDNA towards the first portion of the flow cell that includes the protein.

In some examples, the method further includes removing the positive charges from the lawn primers after interaction between the ligand and the protein.

In some examples, the positive charges are removed through cleavage. In some examples, the positive charges are removed through melt-off.

In some examples, the protein is streptavidin and the ligand is biotin.

In some examples, the lawn primers include P5 lawn primers. In some examples, the lawn primers include P7 lawn primers. In some examples, the lawn primers include P5 and P7 lawn primers.

In some examples, the flow cell does not include any wells on its surface. In some examples, the flow cell includes wells on its surface. In some examples, the wells include small wells within large wells.

In some examples, seeding the dsDNA onto the flow cell inhibits additional seeding events onto the flow cell.

In some examples, the first portion of the flow cell is circular. In some examples, the first portion of the flow cell includes a diameter that is between 80 nm and 100 nm. In some examples, the first portion of the flow cell is a circular pad.

Some examples herein provide a method of seeding double-stranded DNA (dsDNA) onto a flow cell, the method includes seeding the dsDNA onto the flow cell that includes a first portion that includes a first biological molecule and a second portion that includes a plurality of lawn primers immobilised on the flow cell, wherein the dsDNA includes a target nucleic acid sequence, and wherein the dsDNA is connected to a second biological molecule that results in an interaction between the second biological molecule and the first biological molecule.

In some examples, the first biological molecule and the second biological molecule interact through a covalent interaction. In some examples, the first biological molecule and the second biological molecule interact through a non-covalent interaction. In some examples, the non-covalent interaction includes a protein-ligand interaction. In some examples, the protein-ligand interaction includes a streptavidin-biotin interaction.

Some examples herein provide a flow cell, including a surface including a first portion including a pad that includes a protein; and a second portion that includes at least one of P5 lawn primers and P7 lawn primers.

In some examples, the second portion includes both P5 and P7 lawn primers.

In some examples, the at least one of P5 lawn primers and P7 lawn primers are capped with positive charges.

In some examples, the surface includes a patterned surface. In some examples, the patterned surface includes at least one well. In some examples, the at least one well includes at least one well that is contained within at least one large well.

In some examples, the patterned surface includes a gel. In some examples, the gel includes poly(N-(5-azidoacetamidylpentyl) acrylamide-co-acrylamide) (PAZAM).

Some examples herein provide a flow cell, including a surface coated with primers that are capped with a positive charge; and a pad including a protein, wherein the pad has a higher binding energy for dsDNA than the primers that are capped with a positive charge.

In some examples, the positive charge creates a lower energy state at the surface of the flow cell.

In some examples, the protein includes streptavidin.

In some examples, the positive charge is cleavable such that it can be removed from the primer.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIG. 1 schematically illustrates an example of a pad in which the center is coated with streptavidin, and the area around the center is coated with a gel and is grafted with P5/P7 primers.

FIG. 2 schematically illustrates an example of double-stranded DNA that contains a biotin tag.

FIGS. 3A-3C schematically illustrate examples of flow cell surfaces. FIG. 3A schematically illustrates an example embodiment of a flow cell surface with no well structures.

FIG. 3B schematically illustrates an example embodiment of a flow cell surface that includes a small well. FIG. 3C schematically illustrates an example embodiment of a flow cell surface that includes a small well within a large well.

FIG. 4 schematically illustrates an example of surface primers on a flow cell that include cleavable positive charges on the ends of the lawn primers. Because dsDNA has a large exclusion volume than ssDNA, there is a reduction in polyclonality. The cleavable positive charges on the surface primers promote dsDNA migration towards the surface of the flow cell.

FIGS. 5A-5B provide example histograms demonstrating seeding of monoclonal fractions of dsDNA on nanowells. The histogram in FIG. 5A assumes that a 15% overlap between the template and the well is required for the binding to occur. The histogram in FIG. 5B assumes that a 30% overlap between the template and the well is required for the binding to occur.

FIG. 6 demonstrates an example sampling using a Monte Carlo Simulation.

FIGS. 7A-7D schematically illustrate example fabrication flows. FIG. 7A schematically illustrates an example fabrication flow that lacks a well. FIG. 7B schematically illustrates an example fabrication flow in which there is a single well. FIG. 7C schematically illustrates an example fabrication flow in which there is a double well. FIG. 7D schematically illustrates an example fabrication flow that includes a polishing step prior to the PZM deposition.

FIGS. 8A-8B schematically illustrate example groups that are attached on the end of lawn primers that create a binding energy funnel. FIG. 8A schematically illustrates a lawn primer that is capped with a positive charge group in which the positive charge is cleavable. FIG. 8B schematically illustrates a lawn primer that is capped with a positive charge that can be removed by melt-off.

FIGS. 9A-9C schematically illustrate an example operation of a binding energy funnel. FIG. 9A schematically illustrates an example of template strands that preferentially diffuse towards the surface via attraction to a positive charge. FIG. 9B schematically illustrates capture of template strands on a streptavidin pad. FIG. 9C schematically illustrates how lawn primers that contain a positive charge create a lower energy state near the surface of the flow cell relative.

FIG. 10 schematically illustrates an example of a method of monoclonal clustering using dsDNA.

FIGS. 11A-11C schematically illustrate double-stranded DNA template seeding in which primers are capped with a positive charge.

### DETAILED DESCRIPTION

Examples provided herein are methods of monoclonal clustering that are compatible with current library prep protocols.

For example, as provided herein, a flow cell surface is provided that is capable of clustering and seeding. Seeding can be done on pads that include a protein. The clustering can be done in an area of the flow cell that is coated with a gel and that contains primers. The primers can be standard P5 and P7 primers. Template double-stranded DNA (dsDNA) can be labeled with a ligand that can bind to the protein. The dsDNA can attach to the surface of the pad through the interaction of the ligand with the protein. Because the DNA is dsDNA, instead of single-stranded DNA (ssDNA), it can potentially limit additional seeding events on the pad.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes have polynucleotide strands that disassociate from one another. Polynucleotides that are “partially” hybridized to one another means that they have sequences that are complementary to one another, but such sequences are hybridized with one another along only a portion of their lengths to form a partial duplex. Polynucleotides with an “inability” to hybridize include those which are physically separated from one another such that an insufficient number of their bases may contact one another in a manner so as to hybridize with one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primed single stranded target polynucleotide, and can sequentially add nucleotides to the growing primer to form a “complementary copy” polynucleotide having a sequence that is complementary to that of the target polynucleotide. Another polymerase, or the same polymerase, then can form a copy of the target nucleotide by forming a complementary copy of that complementary copy polynucleotide. Any of such copies may be referred to herein as “amplicons.” DNA polymerases may bind to the target polynucleotide and then move down the target polynucleotide sequentially adding nucleotides to the free hydroxyl group at the 3′ end of a growing polynucleotide strand (growing amplicon). DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase. Exemplary polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adapter” that hybridizes to (has a sequence that is complementary to) a primer, and may be amplified so as to generate a complementary copy polynucleotide by adding nucleotides to the free 3′ OH group of the primer. A “capture primer” is intended to mean a primer that is coupled to the substrate and may hybridize to a second adapter of the target polynucleotide, while an “orthogonal capture primer” is intended to mean a primer that is coupled to the substrate and may hybridize to a first adapter of that target polynucleotide. The first adapter may have a sequence that is complementary to that of the orthogonal capture primer, and the second adapter may have a sequence that is complementary to that of the capture primer. A capture primer and an orthogonal capture primer may have different and independent sequences than one another. Additionally, a capture primer and an orthogonal capture primer may differ from one another in at least one other property. For example, the capture primer and the orthogonal capture primer may have different lengths than one another; either the capture primer or the orthogonal capture primer may include a non-nucleic acid moiety (such as a blocking group or excision moiety) that the other of the capture primer or the orthogonal capture primer lacks; or any suitable combination of such properties.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, substrates may include silicon, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface comprising glass or a silicon-based polymer. In some examples, the substrates may include a metal. In some such examples, the metal is gold. In some examples, the substrate has at least one surface comprising a metal oxide. In one example, the surface includes a tantalum oxide or tin oxide. Acrylamides, enones, or acrylates may also be utilized as a substrate material or component. Other substrate materials may include, but are not limited to gallium arsenide, indium phosphide, aluminum, ceramics, polyimide, quartz, resins, polymers and copolymers. In some examples, the substrate and/or the substrate surface may be, or include, quartz. In some other examples, the substrate and/or the substrate surface may be, or include, semiconductor, such as GaAs or ITO. The foregoing lists are intended to be illustrative of, but not limiting to the present application. Substrates may include a single material or a plurality of different materials. Substrates may be composites or laminates. In some examples, the substrate includes an organo-silicate material. Substrates may be flat, round, spherical, rod-shaped, or any other suitable shape. Substrates may be rigid or flexible. In some examples, a substrate is a bead or a flow cell.

In some examples, a substrate includes a patterned surface. A “patterned surface” refers to an arrangement of different regions in or on an exposed layer of a substrate. For example, one or more of the regions may be features where one or more capture primers are present. The features can be separated by interstitial regions where capture primers are not present. In some examples, the pattern may be an x-y format of features that are in rows and columns. In some examples, the pattern may be a repeating arrangement of features and/or interstitial regions. In some examples, the pattern may be a random arrangement of features and/or interstitial regions. In some examples, substrate includes an array of wells (depressions) in a surface. The wells may be provided by substantially vertical sidewalls. Wells may be fabricated as is generally known in the art using a variety of techniques, including, but not limited to, photolithography, stamping techniques, molding techniques and microetching techniques. As will be appreciated by those in the art, the technique used will depend on the composition and shape of the array substrate.

The features in a patterned surface of a substrate may include wells in an array of wells (e.g., microwells or nanowells) on glass, silicon, plastic or other suitable material(s) with patterned, covalently-linked gel such as poly(N-(5-azidoacetamidylpentyl) acrylamide-co-acrylamide) (PAZAM or PZM). The process creates gel pads used for sequencing that may be stable over sequencing runs with a large number of cycles. The covalent linking of the polymer to the wells may be helpful for maintaining the gel in the structured features throughout the lifetime of the structured substrate during a variety of uses. However in many examples, the gel need not be covalently linked to the wells. For example, in some conditions silane free acrylamide (SFA) which is not covalently attached to any part of the structured substrate, may be used as the gel material.

In particular examples, a structured substrate may be made by patterning a suitable material with wells (e.g. microwells or nanowells), coating the patterned material with a gel material (e.g., PAZAM, SFA or chemically modified variants thereof, such as the azidolyzed version of SFA (azido-SFA)) and polishing the surface of the gel coated material, for example via chemical or mechanical polishing, thereby retaining gel in the wells but removing or inactivating substantially all of the gel from the interstitial regions on the surface of the structured substrate between the wells. Primers may be attached to gel material. A solution including a plurality of target polynucleotides (e.g., a fragmented human genome or portion thereof) may then be contacted with the polished substrate such that individual target polynucleotides will seed individual wells via interactions with primers attached to the gel material; however, the target polynucleotides will not occupy the interstitial regions due to absence or inactivity of the gel material. Amplification of the target polynucleotides may be confined to the wells because absence or inactivity of gel in the interstitial regions may inhibit outward migration of the growing cluster. The process is conveniently manufacturable, being scalable and utilizing conventional micro- or nano-fabrication methods.

A patterned substrate may include, for example, wells etched into a slide or chip. The pattern of the etchings and geometry of the wells may take on a variety of different shapes and sizes, and such features may be physically or functionally separable from each other. Particularly useful substrates having such structural features include patterned substrates that may select the size of solid particles such as microspheres. An example patterned substrate having these characteristics is the etched substrate used in connection with BEAD ARRAY technology (Illumina, Inc., San Diego, CA).

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

As used herein, the term “immobilized” when used in reference to a polynucleotide is intended to mean direct or indirect attachment to a substrate via covalent or non-covalent bond(s). In certain examples, covalent attachment may be used, or any other suitable attachment in which the polynucleotides remain stationary or attached to a substrate under conditions in which it is intended to use the substrate, for example, in polynucleotide amplification or sequencing. Polynucleotides to be used as capture primers or as target polynucleotides may be immobilized such that a 3′-end is available for enzymatic extension and at least a portion of the sequence is capable of hybridizing to a complementary sequence. Immobilization may occur via hybridization to a surface attached oligonucleotide, in which case the immobilized oligonucleotide or polynucleotide may be in the 3′-5′ orientation. Alternatively, immobilization may occur by means other than base-pairing hybridization, such as covalent attachment.

As used herein, the term “array” refers to a population of substrate regions that may be differentiated from each other according to relative location. Different molecules (such as polynucleotides) that are at different regions of an array may be differentiated from each other according to the locations of the regions in the array. An individual region of an array may include one or more molecules of a particular type. For example, a substrate region may include a single target polynucleotide having a particular sequence, or a substrate region may include several polynucleotides having the same sequence (or complementary sequences thereof). The regions of an array respectively may include different features than one another on the same substrate. Exemplary features include without limitation, wells in a substrate, beads (or other particles) in or on a substrate, projections from a substrate, ridges on a substrate or channels in a substrate. The regions of an array respectively may include different regions on different substrates than each other. Different molecules attached to separate substrates may be identified according to the locations of the substrates on a surface to which the substrates are associated or according to the locations of the substrates in a liquid or gel. Exemplary arrays in which separate substrates are located on a surface include, without limitation, those having beads in wells.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above exemplary ranges. Exemplary polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A “partially” double stranded polynucleotide may have at least about 10%, at least about 25%, at least about 50%, at least about 60%, at least about 70%, at least about 80%, at least about 90% or at least about 95% of its nucleotides, but fewer than all of its nucleotides, hydrogen bonded to nucleotides in a complementary polynucleotide.

As used herein, the term “single-stranded,” when used in reference to a polynucleotide, means that essentially none of the nucleotides in the polynucleotide are hydrogen bonded to a respective nucleotide in a complementary polynucleotide. A polynucleotide that has an “inability” to hybridize to another polynucleotide may be single-stranded.

As used herein, the term “target polynucleotide” is intended to mean a polynucleotide that is the object of an analysis or action. The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an adapter that functions as a primer binding site, that flank(s) a target polynucleotide sequence that is to be analyzed. A target polynucleotide hybridized to a capture primer may include nucleotides that extend beyond the 5′ or 3′ end of the capture oligonucleotide in such a way that not all of the target polynucleotide is amenable to extension. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “amplicon,” when used in reference to a polynucleotide, is intended to mean a product of copying the polynucleotide, wherein the product has a nucleotide sequence that is substantially the same as, or is substantially complementary to, at least a portion of the nucleotide sequence of the polynucleotide. “Amplification” and “amplifying” refer to the process of making an amplicon of a polynucleotide. A first amplicon of a target polynucleotide may be a complementary copy. Additional amplicons are copies that are created, after generation of the first amplicon, from the target polynucleotide or from the first amplicon. A subsequent amplicon may have a sequence that is substantially complementary to the target polynucleotide or is substantially identical to the target polynucleotide. It will be understood that a small number of mutations (e.g., due to amplification artifacts) of a polynucleotide may occur when generating an amplicon of that polynucleotide.

A substrate region that includes substantially only amplicons of a given polynucleotide may be referred to as “monoclonal,” while a substrate region that includes amplicons of polynucleotides having different sequences than one another may be referred to as “polyclonal.” A substrate region that includes a sufficient number of amplicons of a given polynucleotide to be used to sequence that polynucleotide maybe referred to as “functionally monoclonal.” Illustratively a substrate region in which about 60% or greater of the amplicons are of a given polynucleotide may be considered to be “functionally monoclonal.” Additionally, or alternatively, a substrate region from which about 60% or more of a signal is from amplicons of a given polynucleotide may be considered to be “functionally monoclonal.” A polyclonal region of a substrate may include different subregions therein that respectively are monoclonal. Each such monoclonal region, whether within a larger polyclonal region or on its own, may correspond to a “cluster” generated from a “seed.” The “seed” may refer to a single target polynucleotide, while the “cluster” may refer to a collection of amplicons of that target polynucleotide.

As used herein, the term “biological molecule” is any compound that is found in a living organism and that is capable of carrying out a biological process. A “biological molecule” can be a macromolecule or a small molecule. A “biological molecule” can be a molecule that is capable of binding to another compound, such as another “biological molecule.” These binding interactions can include binding between two (2) macromolecules, binding between two (2) small molecules, or binding between a macromolecule and a small molecule. Examples of binding events include binding between two (2) proteins or binding between a protein and a ligand.

Some examples described herein include a flow cell that contains both a “small well” and a “large well” (e.g., a flow cell that contains a double well). The term “small well” refers to a well in which seeding of double-stranded DNA takes place. In some examples, the length of the surface of a “small well” is equal to or less than about two times the length of the radius of gyration of double-stranded DNA that is being seeded on the surface. In some examples, a “small well” can contain a pad on which seeding takes place that, in some examples, is about the same length as the well. The term “large well” refers to a well that is larger than the “small well” in the flow cell. The “large well” is the well in which clustering takes place.

### Methods of Monoclonal Clustering Using Flow Cells and Double-Stranded DNA

Some examples herein provide a method of amplifying a target nucleic acid sequence, the method includes seeding double-stranded DNA (dsDNA) onto a flow cell that includes a first portion that includes a protein, and second portion that includes a plurality of lawn primers immobilised on the flow cells, wherein the dsDNA includes a target nucleic acid sequence and a complementary nucleic acid sequence, and wherein the dsDNA is connected to a ligand that causes an interaction with the protein; after interaction between the ligand and the protein, denaturing the dsDNA to remove the complementary nucleic acid sequence; and amplifying the target nucleic acid sequence using the lawn primers.

In some examples, seeding the dsDNA onto the flow cells prevents other seeding events on the flow cell. In some examples, seeding the dsDNA onto the flow cell inhibits additional seeding events onto the flow cell. In some examples, it is the size of the dsDNA that prevents other seeding events.

In some examples, the flow cell includes a pad, and the seeding of the dsDNA takes place on the pad. In some examples, the protein on the flow cell is on the pad. In some examples, the pad is coated with a gel. In some examples, the gel includes PAZAM. In some examples, the gel includes any gel described herein.

In some examples, the plurality of lawn primers are capped on one end with positive charges. In some examples, the positive charges are reversibly bound to the lawn primers. In some examples, the method further includes removing the positive charges from the lawn primers after interaction between the ligand and the protein. In some examples, the positive charges are removed through cleavage. In some examples, the positive charges are removed through melt-off.

In some examples, the positive charges cause diffusion of the dsDNA towards the first portion of the flow cell that includes the protein.

In some examples, the method further includes migrating the dsDNA to the surface of the flow cell. In some examples, removal of the positive charge facilitates the migrating step.

In some examples, the protein is streptavidin. In some examples, the ligand is biotin. In some examples, the protein is glutathione-s-transferase. In some examples, the ligand is glutathione. In some examples, the protein is maltose binding protein. In some examples, the ligand is maltose. In some examples, the protein and ligand are any protein and ligand capable of interacting with each other.

In some examples, the lawn primers include P5 lawn primers. In some examples, the lawn primers include P7 lawn primers. In some examples, the lawn primers include P5 and P7 lawn primers.

In some examples, the flow cell does not include any wells on its surface. In some examples, the flow cell includes at least one well on its surface. In some examples, the flow cell includes at least one small well within at least one large well, on the surface of the flow cell.

In some examples, the first portion of the flow cell is circular. In some examples, the first portion of the flow cell includes a circular pad. In some examples, the circular pad includes a diameter that is between about 80 nm and about 100 nm. In some examples, the diameter is less than about 80 nm. In some examples, the diameter is greater than about 100 nm.

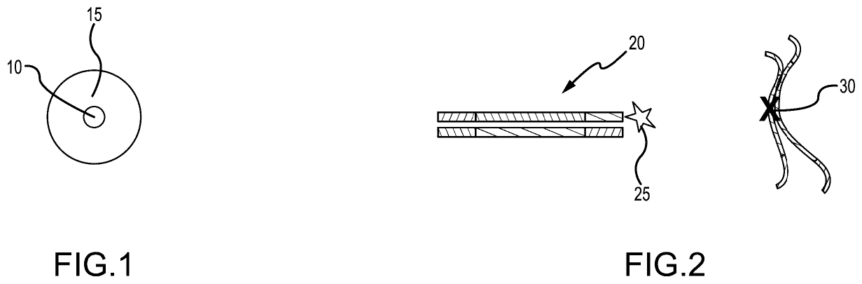

FIG. 1 illustrates an example of a flow cell surface that contains a pad that is coated in a protein 10 and an area around the pad that contains lawn primers. In some examples, the protein is a receptor. In some examples the protein is streptavidin. In some examples, the protein is any protein capable of binding to a ligand. In some examples, the lawn primers include P5 primers, P7 primers, or a combination of P5 primers and P7 primers. FIG. 2 illustrates a double stranded DNA 20 that is connected to a ligand 25. In some examples, the ligand is any ligand capable of binding to a receptor. In some examples, the ligand includes biotin.

Some examples herein provide a method of seeding dsDNA onto a flow cell, the method includes seeding double-stranded DNA (dsDNA) onto the flow cell that includes a first portion that includes a biological molecule and a second portion that includes a plurality of lawn primers immobilised on the flow cells, wherein the dsDNA includes a target nucleic acid sequence, and wherein the dsDNA is connected to a second biological molecule that results in an interaction between the second biological molecule and the first biological molecule.

In some examples, the first biological molecule and the second biological molecule interact through a covalent interaction. In some examples, the first biological molecule and the second biological molecule interact through a non-covalent interaction. In some examples, the non-covalent interaction includes a protein-ligand interaction. In some examples, the protein-ligand interaction includes a streptavidin-biotin interaction.

In some examples, detecting the interaction between the first biological molecule and the second biological molecule includes a sequencing reaction. In some examples, the sequencing reaction includes an amplification reaction. In some examples, the amplification reaction includes bridge amplification or ex-amp.

### Flow Cells

Some examples herein provide a flow cell, which includes a surface that includes a first portion including a pad that is coated with a protein; and a second portion coated with at least one of P5 lawn primers and P7 lawn primers.

In some examples, the second portion is coated with both P5 and P7 lawn primers. In some examples, the at least one of P5 lawn primers and P7 lawn primers are covered with a positive charge.

In some examples, the positive charge has a specific size such that it neutralizes the repulsive force of the negative charges on the lawn primers. In some examples, the positive charge has a specific length such that it neutralizes the repulsive force of the negative charges on the lawn primers.

In some examples, the positive charge includes a molecule that includes multiple positive charges. In some examples, the molecule that includes the multiple positive charges is polylysine, polyspermine, polyethyleneimine and similar molecules.

In some examples, the positive charge can be removed from the primers through cleaving the positive charge from the primers. In some examples, the positive charge can be removed from the primers through melt-off.

In some examples, the surface is made of glass. In some examples, the surface is made of silicon. In some examples, the surface is made of plastic. In some examples the surface includes a patterned surface. In some examples, the patterned surface includes at least one well. In some examples, the at least one well includes at least one small well that is contained within at least one large well.

In some examples, the patterned surface includes a gel. In some examples, the gel includes poly(N-(5-azidoacetamidylpentyl) acrylamide-co-acrylamide) (PAZAM). In some examples, the gel includes SFA. In some examples, the gel includes azido-SFA.

In some examples, the P5 and P7 primers that are coated on the second portion are attached to the gel.

In some examples, the protein that is coated on the first portion is streptavidin. In some examples, the protein that is coated on the first portion is any protein capable of binding to a ligand.

Some examples herein provide a flow cell, including a pad that is (i) coated with a protein and (ii) coated with primers, wherein the protein and the primers are separated. In some examples, the primers are P5 primers, P7 primers, or a combination of P5 and P7 primers.

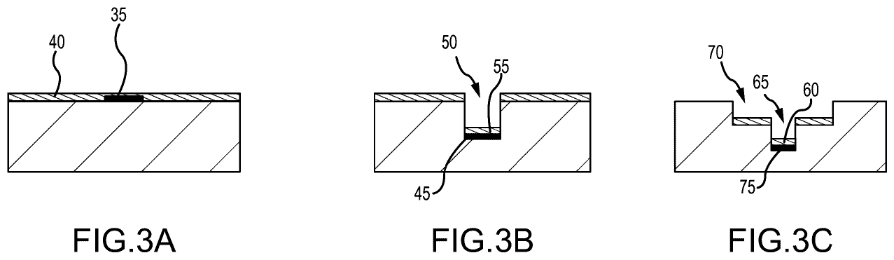

FIG. 3A represents an example embodiment of a flow cell that contains no wells. The flow cell surface 40 contains a small binding pad 35. FIG. 3B represents an example embodiment of a flow cell that contains one (1) well. A small binding pad 45 is on the flow cell surface 55 within the well structure 50. FIG. 3C represents an example embodiment of a flow cell that contains a small well 65 within a large well 70. A small binding pad 75 is on the flow cell surface 60 within the small well. In any of the flow cells exemplified in FIGS. 3A-3C, the small binding pad can be coated with a protein. The protein can be a receptor, or any other protein capable of binding to a ligand. In some examples, the protein is streptavidin. In any of the flow cells exemplified in FIGS. 3A-3C, the flow cell surface can be coated with primers such as P5 primers, P7 primers, or a combination of P5 and P7 primers.

Some examples herein provided a flow cell, including a surface coated with primers that are capped a positive charge; and a pad coated with a protein, wherein the pad has a higher binding energy for dsDNA than the primers that are capped with a positive charge.

In some examples, the pad is circular. In some examples, the circular pad is at least 50 nm in diameter. In some examples, the circular pad is at least about 60 nm in diameter. In some examples, the circular pad is at least about 70 nm in diameter. In some examples, the circular pad is at least about 80 nm in diameter. In some examples, the circular pad is at least about 90 nm in diameter. In some examples, the circular pad is at least about 100 nm in diameter. In some examples, the circular pad is at least about 110 nm in diameter. In some examples, the circular pad is at least about 120 nm in diameter. In some examples, the circular pad is at least about 130 nm in diameter. In some examples, the circular pad is at least about 130 nm in diameter. In some examples, the circular pad is at least about 140 nm in diameter. In some examples, the circular pad is at least about 150 nm in diameter. In some examples, the circular pad is less than about 50 nm in diameter. In some examples, the circular pad is greater than about 150 nm in diameter.

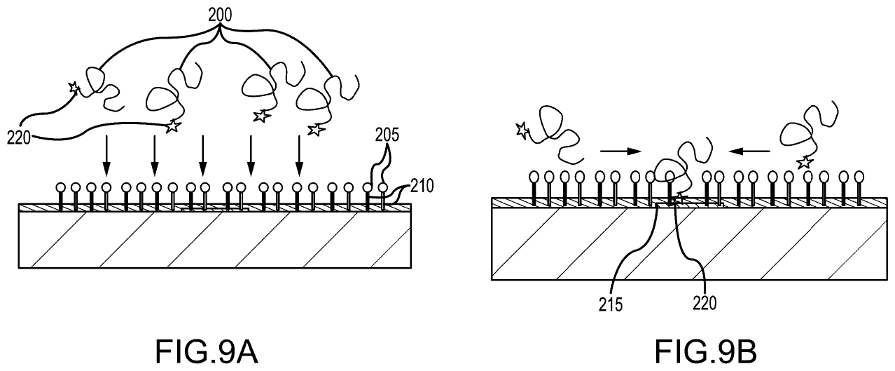

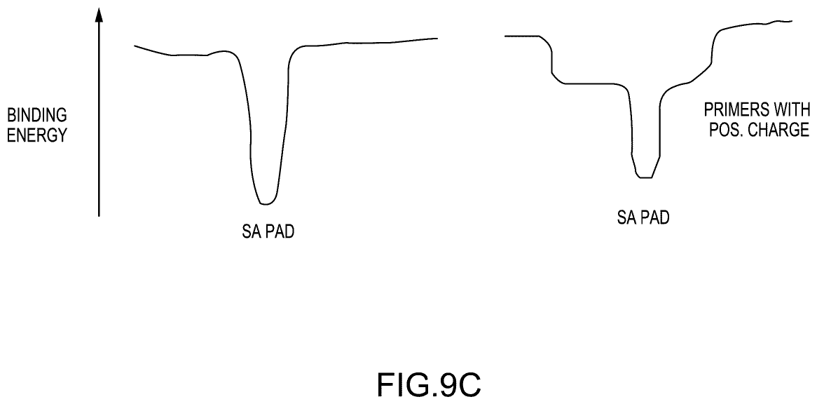

In some examples, the protein is streptavidin. In some examples, the positive charge creates a lower energy state at the surface of the pad that is labeled with the streptavidin. FIG. 9C illustrates an example in which a positive charge results in a lower energy state at the surface of a pad that is coated with streptavidin (labeled SA pad). In some examples, the positive charge is cleavable such that it can be removed from the primer.

As shown in FIG. 9A, the positive charge on the primers can create a binding energy funnel in which dsDNA that includes template strands 200 that are bound to a ligand 220 (e.g. biotin) preferentially diffuses to the surface of the flow cell via attraction to the positive charge 205 on the lawn primers 210. FIG. 9B illustrates that as the template strands diffuse near the surface, the dsDNA attaches to the template/dsDNA through interaction of the streptavidin pad 215 with the ligand 220.

### Fabrication Flows

Various methods of fabrication flows can be used to pattern flow cells.

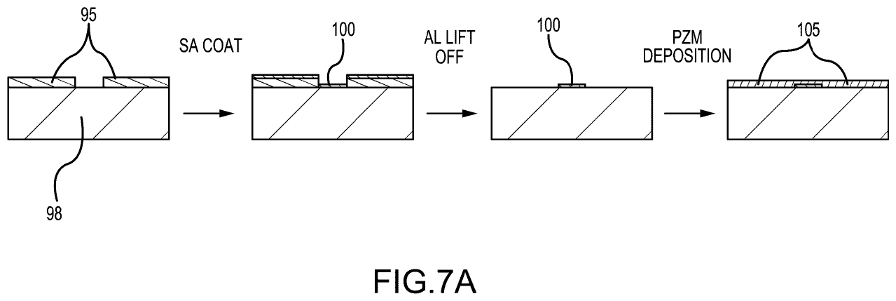

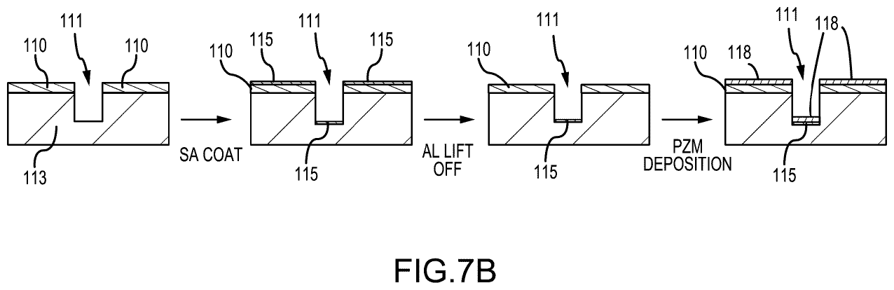

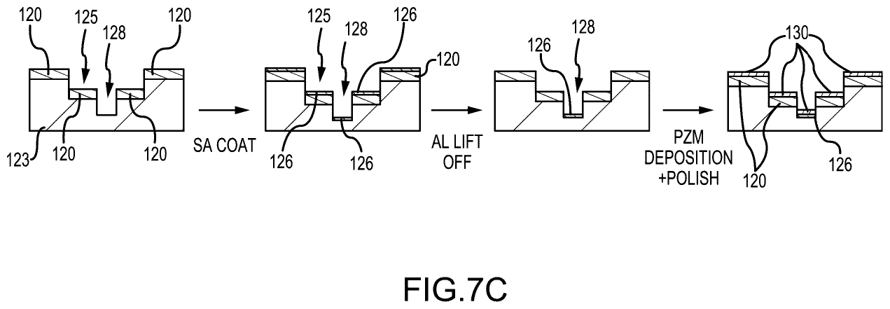

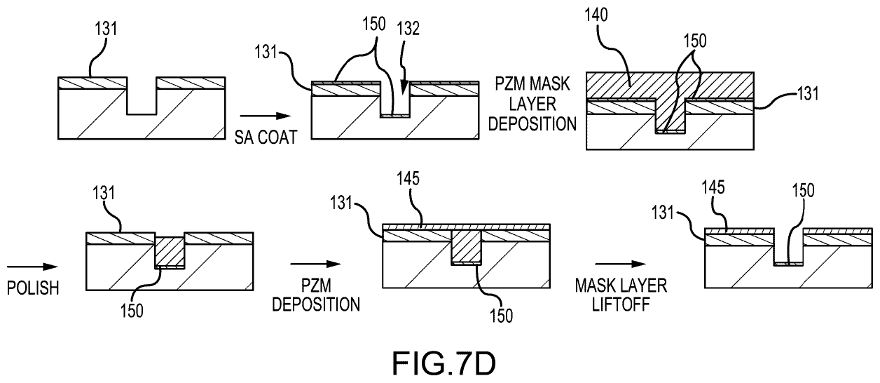

FIG. 7A schematically illustrates an example fabrication flow that lacks a well. A patterned surface 95 can be on a material 98 such as glass, silicon, plastic, or any other suitable material. The patterned surface is coated with streptavidin (SA) 100, or other suitable protein. The surface can be coated with streptavidin, other suitable protein, using a liquid-phase “dunk”, a spin coat, or a droplet dispense.

The flow cell contains a patterned lift-off mask. The mask may be made up of metal such as, for example, aluminum, steel, iron, copper, brass, zinc, bronze, or magnesium. The metal can be patterned via lithography and etching. The thickness of the mask can be between about 50 nm and about 150 nm. In some cases the thickness of the mask is less than 50 nm. In some cases the thickness of the mask is greater than about 150 nm.

After a lift off step, the protein coated (e.g., streptavidin-coated) patterned surface is left behind 100. The lift off step is performed with a chemical that is capable of removing the metal (e.g., aluminum), but does not remove the protein (e.g., streptavidin). The surface is then coated with PAZAM 105, or other suitable gel material.

FIG. 7B schematically illustrates an example fabrication flow in which there is a single well 111. A patterned surface 110 can be on a material 113 such as glass, silicon, plastic, or any other suitable material. The patterned surface is coated with protein such as streptavidin 115 which results in coating the well 111 with the protein (e.g., streptavidin) 115. The surface can be coated with protein (e.g., streptavidin) using a liquid-phase “dunk”, a spin coat, or a droplet dispense.

The flow cell contains a patterned lift-off mask. The mask may be made up of metal such as, for example, aluminum, steel, iron, copper, brass, zinc, bronze, or magnesium. The metal can be patterned via lithography and etching. The thickness of the mask can be between about 50 nm and about 150 nm. In some cases the thickness of the mask is less than about 50 nm. In some cases the thickness of the mask is greater than about 150 nm.

After a lift off step, only the well 111 remains coated with a protein (e.g., streptavidin) 115. The lift off step is performed with a chemical that is capable of removing the metal (e.g., aluminum), but does not remove the protein (e.g., streptavidin) from the well. The patterned surface 110 and the well 111 are then coated with PAZAM 118, or other suitable gel material.

FIG. 7C schematically illustrates an example fabrication flow in which there is a double well, an upper well (large well) 125 and a lower well (small well) 128. A patterned surface 120 can be on a material 123 such as glass, silicon, plastic, or any other suitable material. The patterned surface is coated with a protein (e.g., streptavidin) which results in coating the upper well 125 with the protein (e.g., streptavidin) 126 and the lower well 128 with the protein (e.g., streptavidin) 126. The surface can be coated with the protein (e.g., streptavidin) using a liquid-phase “dunk”, a spin coat, or a droplet dispense.

The flow cell contains a patterned lift-off mask. The mask may be made up of metal such as, for example, aluminum, steel, iron, copper, brass, zinc, bronze, or magnesium. The metal can be patterned via lithography and etching. The thickness of the mask can be between 50 nm and 150 nm. In some cases the thickness of the mask is less than about 50 nm. In some cases the thickness of the mask is greater than about 150 nm.

After a lift off step, only the lower well 128 remains coated with the protein (e.g., streptavidin) 126. The lift off step is performed with a chemical that is capable of removing the metal (e.g., aluminum), but does not remove the protein (e.g., streptavidin). The surface is then coated with PAZAM 130, or other suitable gel material.

FIG. 7D schematically illustrates an example fabrication flow that includes a polishing step prior to the PAZAM deposition (or deposition with another suitable gel material). The patterned surface 131 is coated with a protein (e.g., streptavidin) which results in coating the well 132 with the protein (e.g., streptavidin) 150. The surface can be coated with the protein (e.g., streptavidin) using a liquid-phase “dunk”, a spin coat, or a droplet dispense.

After coating with streptavidin, or other suitable protein, a masking material is deposited 140. An example of masking material is a carbon hardmask. The substrate would then be polished to remove the streptavidin that is coated outside of the well and to remove the mask layer that is deposited outside of the well.

A PAZAM layer, other suitable gel material, is deposited, and the mask layer lifted off. The chemical used for the lift-off would only remove the mask material, while leaving the PAZAM 145, or other suitable gel material, and the protein (e.g., streptavidin) 150 on the well.

### Bridge Amplification

Bridge amplification can occur on a flow cell. Double-stranded template DNA is hybridized to lawn primers in a flow cell, and a polymerase is used to extend the primer to form double-stranded DNA. The double-stranded DNA is denatured, and the original template strand of the DNA molecule is washed away. This results in a single-stranded DNA molecule being bound to the lawn primers of the flow cell. The single-stranded DNA molecule turns over and forms a “bridge” by hybridising to a nearby lawn primer that is complementary to a sequence of the single-stranded DNA molecule. Polymerase extends the hybridized primer resulting in bridge amplification of the DNA molecule and the creation of a double-stranded DNA molecule. The double-stranded DNA molecule is then denatured resulting in two copies of single-stranded templates, one of which is immobilized to the support and the other of which may be washed away. The one which is immobilized as the support may be used in further bridge amplification operations so as to generate a cluster that subsequently may be sequenced.

### Nucleic Acids and Template Libraries

As will be understood by the skilled person, a double-stranded nucleic acid will typically be formed from two complementary polynucleotide strands made up of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g., linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support.

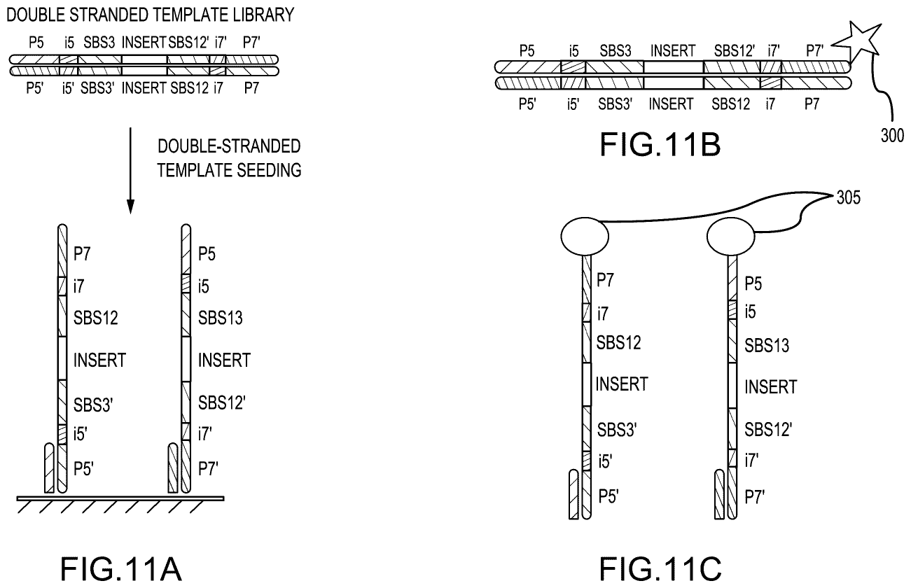

An example of a typical double-stranded nucleic acid template (which may be provided in a library of such templates) is shown in FIG. 11A. In one example, a first strand of the template includes, in the 5′ to 3′ direction, a first lawn primer-binding sequence (e.g., P5), an index sequence (e.g., i5), a first sequencing primer binding site (e.g., SBS3), an insert corresponding to the template DNA to be sequenced, a second sequencing primer binding site (e.g. SBS12′), a second index sequence (e.g. i7′) and a second lawn primer-binding sequence (e.g. the complement of P7). The second strand of the template includes, in the 3′ to 5′ direction, a first lawn primer-binding site (e.g. the complement of P5), an index sequence (e.g. i5′, which is complementary to i5), a first sequencing primer binding site (e.g. SBS3′ which is complementary to SBS3), an insert corresponding to the complement of the template DNA to be sequenced, a second sequencing primer binding site (e.g. SBS12, which is complementary to SBS12), a second index sequence (e.g. i7, which is complementary to i7) and a second lawn primer-binding sequence (e.g. P7).

As shown in FIG. 11B, the double-stranded can include a biotin tag 300, which can be used to bind to a pad coated with streptavidin that is on the surface of the flow cell. The double-stranded DNA can be labelled with the biotin tag during library preparation. As shown in FIG. 11C, the P5 and P7 lawn primers may contain a positively charged molecule 305 on one of its ends.

In some examples, the primer-binding sequences of the adaptors are complementary to short primer sequences (or lawn primers) present on the surface of the flow cells. Binding of suitable portions of the adaptors to their complements (P5 and P7) on—for example—the surface of the flow cell, permits nucleic acid amplification.

The primer-binding sequences in the adaptor which permit hybridisation to amplification (lawn) primers will typically be around 20-40 nucleotides in length, although, in examples, the disclosure is not limited to sequences of this length. The precise identity of the amplification primers, and hence the cognate sequences in the adaptors, are generally not material to the disclosure as long as the primer-binding sequences are able to interact with the amplification primers in order to direct amplification. The sequence of the amplification primers may be specific for a particular target nucleic acid that it is desired to amplify, but in other examples these sequences may be “universal” primer sequences which enable amplification of any target nucleic acid of known or unknown sequence which has been modified to enable amplification with the universal primers. The criteria for design of PCR primers are generally well known to those of ordinary skill in the art. “Primer-binding sequences” may also be referred to as “clustering sequences” “clustering primers” or “cluster primers” in the present disclosure, and such terms may be used interchangeably.

The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analysed in a single run, without drastically increasing run cost or run time. Examples of tag sequences are found in WO05068656, the entire contents of which are incorporated by reference herein. The tag can be read at the end of the first read, or equally at the end of the second read. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example in U.S. Pat. No. 8,822,150, the entire contents of which are incorporated by reference herein. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify, and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e., hybridises) to a portion of the sequencing binding site on the template strand. The DNA polymerase enzyme binds to this site and incorporates complementary nucleotides base by base into the growing opposite strand. In one example, the sequencing process includes a first and second sequencing read. The first sequencing read may include the binding of a first sequencing primer (read 1 sequencing primer) to the first sequencing binding site (e.g., SBS3′) followed by synthesis and sequencing of the complementary strand. This leads to the sequencing of the insert. In a second step, an index sequencing primer (e.g., i7 sequencing primer) binds to a second sequencing binding site (e.g., SBS12) leading to synthesis and sequencing of the index sequence (e.g., sequencing of the i7 primer). The second sequencing read may include binding of an index sequencing primer (e.g., i5 sequencing primer) to the complement of the first sequencing binding site on the template (e.g., SBS3) and synthesis and sequencing of the index sequence (e.g., i5). In a second step, a second sequencing primer (read 2 sequencing primer) binds to the complement of the primer (e.g., i7 sequencing primer) binds to a second sequencing binding site (e.g., SBS12′) leading to synthesis and sequencing of the insert in the reverse direction.

### Solid Supports

The disclosure may make use of solid supports made up of a substrate or matrix (e.g., glass slides, polymer beads etc) which has been “functionalised”, for example by application of a layer or coating of an intermediate material comprising reactive groups which permit attachment to biomolecules, such as polynucleotides. Examples of such supports include, but are not limited to, a substrate such as glass. In such examples, the biomolecules (e.g., polynucleotides) may be directly covalently attached to the intermediate material but the intermediate material may itself be non-covalently attached to the substrate or matrix (e.g., the glass substrate). The term “covalent attachment to a solid support” is to be interpreted accordingly as encompassing this type of arrangement. Alternatively, the substrate such as glass may be treated to permit direct covalent attachment of a biomolecule. In some examples, a pad coated with streptavidin is placed on the solid support. The streptavidin is capable of binding to templates that are connected to biotin.

### Amplification and Sequencing of Template Strands

Once a library comprising template nucleotide strands has been prepared, the templates are seeded onto a solid support and then amplified to generate a cluster of single template molecules.

By way of brief example, following attachment of the P5 and P7 primers, the solid support may be contacted with the template to be amplified under conditions which permit hybridisation (or annealing—such terms may be used interchangeably) between the template and the immobilised primers (also referred to herein as “lawn primers”). The template is usually added in free solution under suitable hybridisation conditions, which will be apparent to the skilled reader. Typically, hybridisation conditions are, for example, 5×SSC at 40° C. Solid-phase amplification can then proceed. The first step of the amplification is a primer extension step in which nucleotides are added to the 3′ end of the immobilised primer using the template to produce a fully extended complementary strand. The template is then typically washed off the solid support. The complementary strand will include at its 3′ end a primer-binding sequence (i.e., the complement of either P5 or P7) which in some methods is capable of bridging to the second primer molecule immobilised on the solid support and binding. In this method, further rounds of amplification (analogous to a standard PCR reaction) lead to the formation of clusters or colonies of template molecules bound to the solid support. Thus, in this example, solid-phase amplification by either the method analogous to that of WO 98/44151 or that of WO 00/18957 (the contents of which are incorporated herein in their entirety by reference) will result in production of a clustered array that includes colonies of “bridged” amplification products. Both strands of the amplification products will be immobilised on the solid support at or near the 5′ end, this attachment being derived from the original attachment of the amplification primers. Typically, the amplification products within each colony will be derived from amplification of a single template (target) molecule. Other amplification procedures may be used, and will be known to the skilled person. For example, amplification may be isothermal amplification using a strand displacement polymerase; or may be exclusion amplification as described in WO 2013/188582, the entire contents of which are incorporated by reference herein. The method may also involve a number of rounds of invasion by a competing immobilised primer (or lawn primer) and strand displacement of the template to the competing primer. Further information on amplification can be found in WO0206456 and WO07107710, the entire contents of each of which are incorporated by reference herein. Through such approaches, a cluster of single template molecules is formed.

Sequence data can be obtained from both ends of a template duplex by obtaining a sequence read from one strand of the template from a primer in solution, copying the strand using immobilised primers, releasing the first strand, and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′-end. If the first strand is removed from the surface, the sequence of the second strand can be read. This gives a sequence read from both ends of the original fragment. The process whereby the strand is regenerated after the first read is known as “Paired-end resynthesis” or “PE resynthesis”. The typical steps of pairwise sequencing are known and have been described in WO 2008/041002, the entire contents of which are incorporated by reference herein.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators include removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application WO 2007/135368 the entire contents of which are incorporated by reference herein. Alternatively, a separate reaction may be carried out including each of the modified nucleotides added individually.

The modified nucleotides may carry a label to facilitate their detection. In a particular example, the label is a fluorescent label. Each nucleotide type may carry a different fluorescent label. However, the detectable label need not be a fluorescent label. Any label can be used which allows the detection of the incorporation of the nucleotide into the DNA sequence. One method for detecting the fluorescently labelled nucleotides includes using laser light of a wavelength specific for the labelled nucleotides, or the use of other suitable sources of illumination. The fluorescence from the label on an incorporated nucleotide may be detected by a CCD camera or other suitable detection means. Suitable detection means are described in WO 2007/123744, the entire contents of which are incorporated by reference herein.

Alternative methods of sequencing include sequencing by ligation, for example as described in U.S. Pat. No. 6,306,597 or WO06084132, the entire contents of each of which are incorporated by reference herein.

An extension reaction, in which nucleotides are added to the 3′ end of a primer is performed using a polymerase, such as a DNA or RNA polymerase. In one example, the polymerase is a non-thermal isothermal strand displacement polymerase. Suitable non-thermostable strand displacement polymerases according to the present disclosure can be found, for example, through New England BioLabs, Inc. and include phi29, Bsu, Klenow, DNA Polymerase I (E. coli), and Therminator. A particularly preferred polymerase is Bsu.

Reference to P5 and P7 could refer to different primer sequences. Any suitable primer sequence combinations are encompassed by the present disclosure.

### Examples

The following representative examples are representative of the embodiments described herein and are not meant to be limiting in any way.

### Example 1. A Method of Monoclonal Clustering on Flow Cells

The flow cell surface is prepared with orthogonal chemistries for clustering and seeding. As shown in FIG. 1, seeding can only be done on pads coated in streptavidin 10, whereas clustering can take place in the area that is coated with PAZAM grafted with the standard P5/P7 chemistry 15.

As shown in FIG. 2, template DNA 20 is labeled with ligand such as biotin 25 during library prep to enable seeding on the streptavidin pads. The library is left as double-stranded DNA (dsDNA), which has a much larger radius of gyration than single-stranded DNA. Because the DNA is dsDNA, non-specific and self-interactions are blocked 30.

As a result, seeding leads to size exclusion that reduces the probability that multiple seeding events take place for a single cluster. That is, once a streptavidin patch has been seeded by a single dsDNA template molecule, the large footprint of the molecule tends to limit additional seeding events on the same pad.

dsDNA seeding reduces (1) non-specific interactions between ssDNA molecules that can lead to bunching of library molecules, which can lead to non-Poissonian seeding biased towards polyclonal clusters and (2) non-specific self-interactions that lead to secondary DNA structure, which can impede seeding or clustering.

The small binding pad can be formed in a variety of ways. As shown in FIG. 3A, a small binding pad 35 can be formed on a flow cell surface 40 with no well structures. As shown in FIG. 3B, a small binding pad 45 can be formed on a small well structure 50 on a flow cell surface 55. As shown in FIG. 3C, a small binding pad 60 can be formed inside a small well 65 within a large well 70, on a flow cell surface 75.

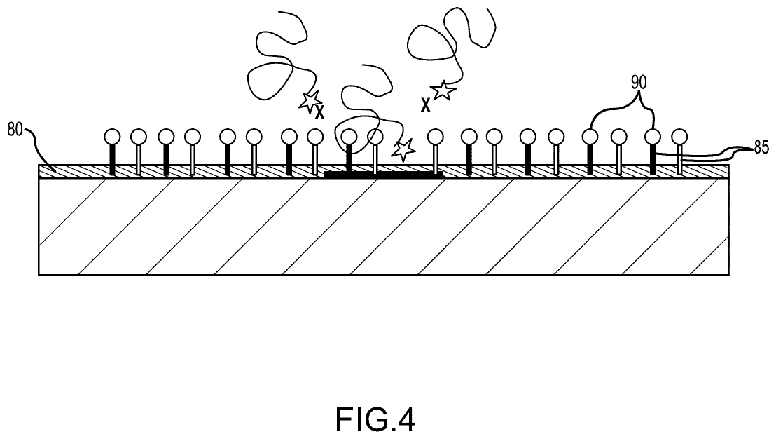

In order to increase the speed at which seeding occurs, this method can optionally be combined with the use of a cleavable, positively charge molecule added to the end of the surface primers. FIG. 4 provides an example of a flow cell surface 80 that contains surface primers 85. Each of the surface primers contain a cleavable positive charge 90 that promotes dsDNA migration towards the surface of the flow cell. Specifically, the positive charge would act as a binding funnel to attract negatively charged dsDNA template strands to the surface, thereby increasing their interaction probability with the streptavidin pads via diffusion.

### Example 2. Effect of Gyration Radius of dsDNA on Monoclonal Fraction

It is known that dsDNA has a larger radius of gyration relative to ssDNA; a 1,000 base pair strand of dsDNA results in about a 70 nm radius (Douglas R. Tree, Abhiram Muralidhar, Patrick S. Doyle, and Kevin D. Dorfman, Is DNA a Good Model Polymer? Macromolecules 46, 20, 8369-8382 (2013)). Due to this radius of gyration, dsDNA has a larger exclusion footprint upon seeding, relative to ssDNA.

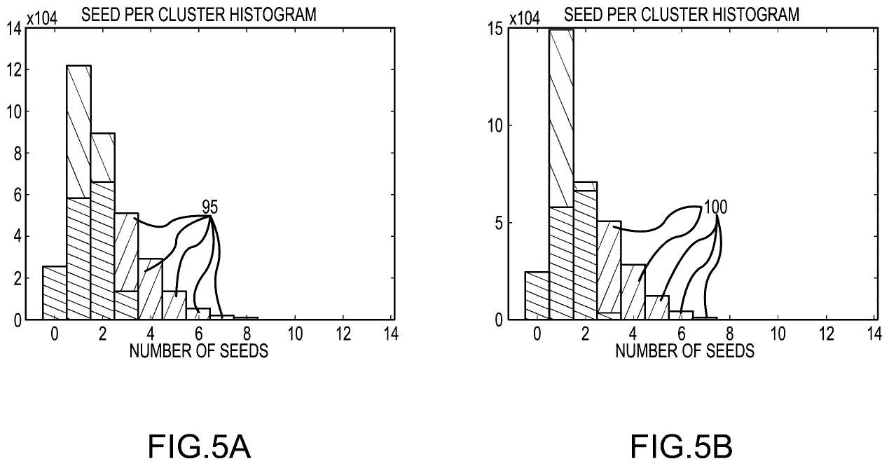

In a simulation, random seeding locations were drawn across a grid of nanowells. An assumption is made that template strands do not overlap. A certain fraction of overlap of template and well is required for binding to occur. The data from the simulation produced in the histograms shown in FIGS. 5A and 5B resulted from using a 70 nm radius of gyration and a 100 nm pad. It is assumed that a 100 nm pad can be used for fabrication, but smaller pads may be possible.

FIG. 5A shows a simulation of seeding in which it is assumed that a 15% overlap between the template and the well is needed. Element 95 shows bar graphs of seeding distribution with no size exclusion. FIG. 5B shows a simulation of a seeding in which it is assumed that a 30% overlap between the template and the well is needed. Element 100 shows bar graphs of seeding distribution with no size exclusion.

The simulation proceeded until 90% of the wells were occupied. If 50 nm pads could be fabricated, simulations suggest that seeding with 70 nm dsDNA could enable 80-90+% of occupied wells to be monoclonal.

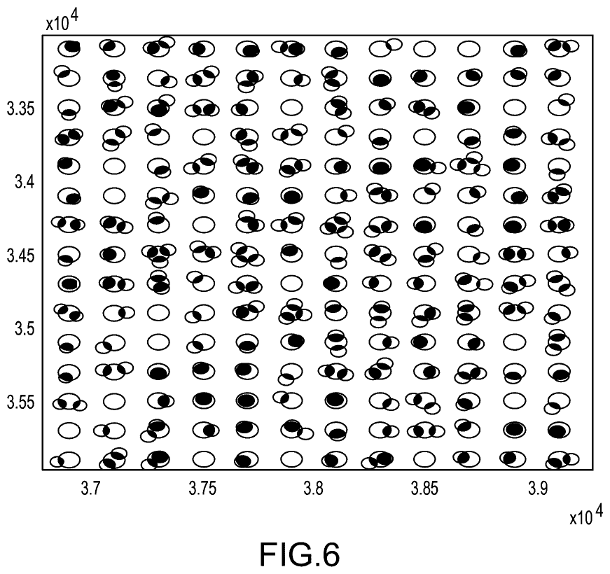

FIG. 6 illustrates a sampling from a Monte Carlo Simulation, which shows the fraction of wells that were monoclonally seeded at various possible parameters such as well radius, DNA radius of gyration, and DNA overlap. Several thousand wells and dsDNA strands were tested. In the simulation, each simulated DNA molecule was assumed to interact on a flow cell at a random location. If the footprint of the DNA (defined by its assumed radius of gyration) lacked sufficient overlap with the cell, it was assumed that the DNA strand was not seeded in the simulation. Also, if the DNA strand only overlapped a well that was already occupied by another DNA strand, it was assumed that the DNA strand was not seeded in the simulation. If, however, the DNA strand overlapped with a well in a location that was not previously seeded, then it was assumed that the DNA strand was seeded in the well where it overlapped. This seeding event blocked other DNA strands from seeding at that well.

Table 1 shows what the percent seeding would be under this model system if there was no size exclusion, if 15% overlap between the template and the well was needed, and if 30% overlap between the template and the well was needed.

TABLE 1

| | 1 seed | 2 Seeds | 3 Seeds | 4+ Seeds |
|---|---|---|---|---|
| No Size | 23.2% | 26.6% | 20.4% | 19.9% |
| Exclusion | | | | |
| 15% Overlap | 48.8% | 35.8% | 5.4% | 0.1% |
| Needed | | | | |
| 30% Overlap | 59.7% | 28.5% | 1.8% | 0% |
| Needed | | | | |

### Example 3. Binding Energy Funnels

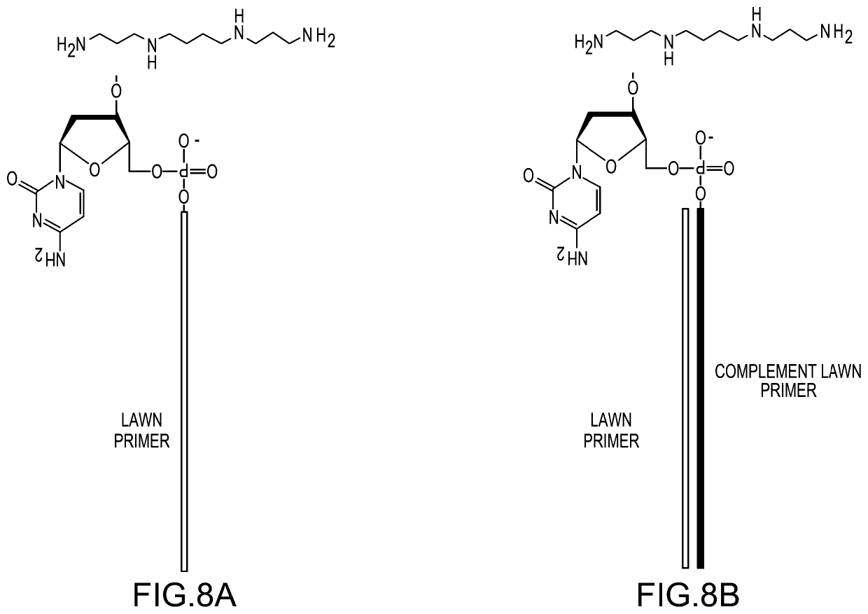

FIG. 8A provides an example of a binding energy funnel in which a cleavable, positively charged group is attached to a lawn primer. Cleavage, for example, can occur through cleaving a disulfide bond. FIG. 8B provides an example of a binding energy funnel in which a complement lawn primer is connected to a positively charged group. The positively charged group can be removed by melt-off.

### Example 4. Mechanism of Action of Binding Energy Funnels

FIGS. 9A and 9B provide an example of a mechanism of action of a binding energy funnel. As shown in FIG. 9A, template strands 200 diffuse (as shown by the arrows) to the surface of the flow cell due to the positive charge 205 on the end of the lawn primers 210. The positive charge on the lawn primers can be molecules of various lengths or molecules of various sizes such that they are able to adequately neutralize the repulsive force of the negative charges of the surface primers. The various sizes and lengths of the molecules are described herein. Molecules such as polylysine that contain multiple positive charges can also be used. Other molecules that contain multiple positive charges are described herein. In addition the ionic strength can also be modulated to reduce electrostatic repulsion. Alternatively the pH of the buffer solution can also be controlled to in turn control the effective positive charge on the surface. As shown in FIG. 9B, as template strands diffuse near the surface, they are captured on streptavidin pad 215 through interaction of the biotin tag 220 with the streptavidin.

The binding energy between biotin and the streptavidin pad is much higher than a weak primer charge interaction. Also, the positive charges on the lawn primer create a lower energy state near the surface of the flow cell relative to the rest of the fluidic channel on the flow cell (FIG. 9C).

### Example 5. An Example Method of Monoclonal Clustering Using dsDNA

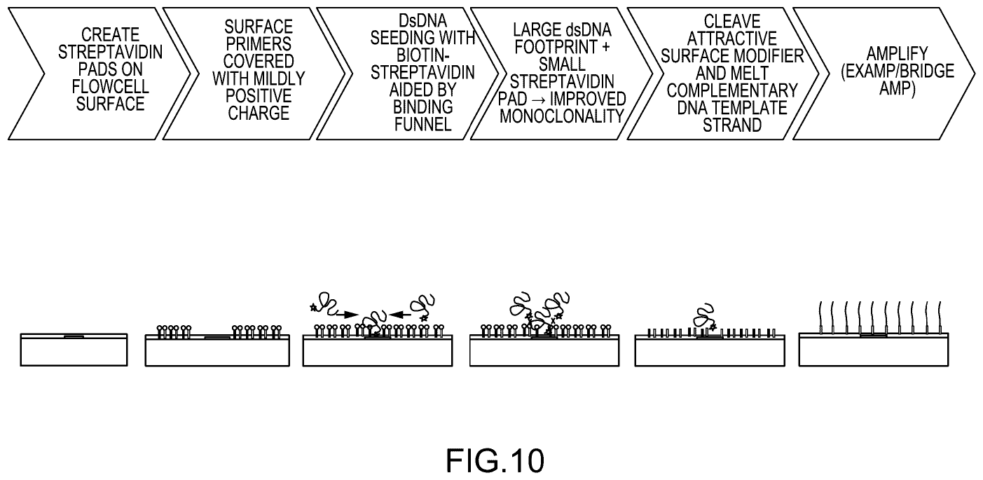

FIG. 10 provides an example method of monoclonal clustering using dsDNA. The method contains the following steps:
1. Streptavidin pads are created on a flow cell surface.
2. Surface primers that are covered with a mildly positive charge are added to the flow cell surface.
3. Double-stranded DNA (dsDNA) that is bound to biotin is seeded on the flow cell surface. The dsDNA contains a target nucleic acid sequence and a complementary nucleic acid sequence. The large dsDNA relative to the smaller sized pad improves monoclonality.
4. The mildly positive charge is removed from the surface primers.
5. The DNA is melted to remove the complementary nucleic acid sequence.
6. The target nucleic acid is sequenced to determine its identity. The target can be amplified through examplification or bridge amplification. Example 5 describes the dsDNA bound to the lawn primers that can be amplified through ex-amp or bridge amplification.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of amplifying a target nucleic acid sequence, the method comprising:
seeding double-stranded DNA (dsDNA) onto a flow cell that comprises a first portion that comprises a protein, and a second portion that comprises a plurality of lawn primers immobilised on the flow cell,
wherein the dsDNA comprises a target nucleic acid sequence and a complementary nucleic acid sequence, and wherein the dsDNA is connected to a ligand that causes an interaction between the ligand and the protein;
after interaction between the ligand and the protein, denaturing the dsDNA to remove the complementary nucleic acid sequence; and
amplifying the target nucleic acid sequence using the lawn primers.

2. The method of claim 1, wherein the plurality of lawn primers are capped on one end with positive charges.

3. The method of claim 2, wherein the positive charges cause diffusion of the dsDNA towards the first portion of the flow cell that comprises the protein.

4. The method of claim 2, further comprising removing the positive charges from the lawn primers after interaction between the ligand and the protein.

5. The method of claim 4, wherein the positive charges are removed through cleavage.

6. The method of claim 4, wherein the positive charges are removed through melt-off.

7. The method of claim 1, wherein the protein is streptavidin, and wherein the ligand is biotin.

8. The method of claim 1, wherein the lawn primers comprise P5 lawn primers.

9. The method of claim 1, wherein the lawn primers comprise P7 lawn primers.

10. The method of claim 1, wherein the lawn primers comprise P5 and P7 lawn primers.

11. The method of claim 1, wherein the flow cell does not include any wells on its surface.

12. The method of claim 1, wherein the flow cell comprises wells on its surface.

13. The method of claim 12, wherein the wells comprise small wells within large wells.

14. The method of claim 1, wherein seeding the dsDNA onto the flow cell inhibits additional seeding events onto the flow cell.

15. The method of claim 1, wherein the first portion of the flow cell is circular.

16. The method of claim 15, wherein the first portion of the flow cell comprises a diameter that is between 80 nm and 100 nm.

17. The method of claim 15, wherein the first portion of the flow cell is a circular pad.

18. A method of seeding double-stranded DNA (dsDNA) onto a flow cell, the method comprising:
seeding the dsDNA onto the flow cell that comprises a first portion that comprises a first biological molecule and a second portion that comprises a plurality of lawn primers immobilised on the flow cell,
wherein the dsDNA comprises a target nucleic acid sequence, and wherein the dsDNA is connected to a second biological molecule that results in an interaction between the second biological molecule and the first biological molecule.

19-22. (canceled)

23. A flow cell, comprising:
a surface, comprising
a first portion comprising a pad that comprises a protein; and
a second portion that comprises at least one of P5 lawn primers and P7 lawn primers.

24-34. (canceled)
